(19) United States
(12) Patent Application Publication
(10) Pub. No.: US 2023/0290498 A1
(43) Pub. Date: Sep. 14, 2023

(54) Autonomous Drone System and Method

(71) Applicant: MAVRIK Technologies LLC, Charlotte, NC (US)

(72) Inventor: Maxwell Holloway Owens, Charlotte, NC (US)

(21) Appl. No.: 18/181,366

(22) Filed: Mar. 9, 2023

Related U.S. Application Data
(60) Provisional application No. 63/318,284, filed on Mar. 9, 2022.
(60) Provisional application No. 63/318,291, filed on Mar. 9, 2022.

(51) Int. Cl. G16H 40/67; B64C 39/02; G10L 15/22; H04W 4/02; H04W 4/40; H04W 4/90

(52) U.S. Cl. CPC G16H 40/67; B64C 39/024; G10L 15/22; H04W 4/025; H04W 4/40; H04W 4/90; B64U 2101/55

(57) ABSTRACT

A computer-implemented method, computer program product and computing system for processing a medical assistance request from a requester; defining an incident location for the medical assistance request; assigning an autonomous drone to the medical assistance request, thus defining an assigned autonomous drone; and dispatching the assigned autonomous drone to the incident location.

### RELATED APPLICATION(S)

This application claims the benefit of U.S. Provisional Application Nos. 63/318,284 filed on 9 Mar. 2022 and 63/318,291 filed on 9 Mar. 2022, the entire contents of which are incorporated herein by reference.

### TECHNICAL FIELD

This disclosure relates to autonomous drone systems and methods and, more particularly, to autonomous drone guidance systems and methods.

### BACKGROUND

The use of drones is exploding around the world. Accordingly, such drones are used to take photographs, record videos, perform survey operations, perform military operations, etc. As such drones continue to advance, the autonomy of such drones is continuously increasing. Accordingly, various companies are using autonomous drones to deliver packages.

Therefore, it is foreseeable that there exists a need to regulate the manner in which such autonomous drones share the airspace with commercial aircraft and navigate around various obstacles.

### SUMMARY OF DISCLOSURE

Automated Search/Rescue/Ambulatory

In one implementation, a computer-implemented method is executed on a computing device and includes: processing a medical assistance request from a requester; defining an incident location for the medical assistance request; assigning an autonomous drone to the medical assistance request, thus defining an assigned autonomous drone; and dispatching the assigned autonomous drone to the incident location.

One or more of the following features may be included. Processing a medical assistance request from a requester may include one or more of: processing the medical assistance request from the requester via a voice-based virtual assistant; processing the medical assistance request from the requester via an application program interface; and processing the medical assistance request from the requester via a chatbot. Defining an incident location for the medical assistance request may include one or more of: obtaining the incident location from the requester; obtaining the incident location from a location database; obtaining the incident location from a GPS chipset included within a handheld electronic device; and obtaining the incident location via cell tower triangulation of a handheld electronic device. Processing a medical assistance request from a requester may include: identifying an incident type for the medical assistance request. Assigning an autonomous drone to the medical assistance request, thus defining an assigned autonomous drone may include: assigning an autonomous drone to the medical assistance request based, at least in part, upon the incident type. The assigned autonomous drone may be configured to transport a medical professional to the incident location. The assigned autonomous drone may be configured to search the incident location for a subject of the medical assistance request. The assigned autonomous drone may be configured to transport a subject of the medical assistance request to a medical facility. The assigned autonomous drone may be configured to communicate with a medical facility. The assigned autonomous drone may be configured to communicate with a subject of the medical assistance request.

In another implementation, a computer program product resides on a computer readable medium and has a plurality of instructions stored on it. When executed by a processor, the instructions cause the processor to perform operations including processing a medical assistance request from a requester; defining an incident location for the medical assistance request; assigning an autonomous drone to the medical assistance request, thus defining an assigned autonomous drone; and dispatching the assigned autonomous drone to the incident location.

One or more of the following features may be included. Processing a medical assistance request from a requester may include one or more of: processing the medical assistance request from the requester via a voice-based virtual assistant; processing the medical assistance request from the requester via an application program interface; and processing the medical assistance request from the requester via a chatbot. Defining an incident location for the medical assistance request may include one or more of: obtaining the incident location from the requester; obtaining the incident location from a location database; obtaining the incident location from a GPS chipset included within a handheld electronic device; and obtaining the incident location via cell tower triangulation of a handheld electronic device. Processing a medical assistance request from a requester may include: identifying an incident type for the medical assistance request. Assigning an autonomous drone to the medical assistance request, thus defining an assigned autonomous drone may include: assigning an autonomous drone to the medical assistance request based, at least in part, upon the incident type. The assigned autonomous drone may be configured to transport a medical professional to the incident location. The assigned autonomous drone may be configured to search the incident location for a subject of the medical assistance request. The assigned autonomous drone may be configured to transport a subject of the medical assistance request to a medical facility. The assigned autonomous drone may be configured to communicate with a medical facility. The assigned autonomous drone may be configured to communicate with a subject of the medical assistance request.

In another implementation, a computing system includes a processor and a memory system configured to perform operations including processing a medical assistance request from a requester; defining an incident location for the medical assistance request; assigning an autonomous drone to the medical assistance request, thus defining an assigned autonomous drone; and dispatching the assigned autonomous drone to the incident location.

One or more of the following features may be included. Processing a medical assistance request from a requester may include one or more of: processing the medical assistance request from the requester via a voice-based virtual assistant; processing the medical assistance request from the requester via an application program interface; and processing the medical assistance request from the requester via a chatbot. Defining an incident location for the medical assistance request may include one or more of: obtaining the incident location from the requester; obtaining the incident location from a location database; obtaining the incident location from a GPS chipset included within a handheld electronic device; and obtaining the incident location via cell tower triangulation of a handheld electronic device. Processing a medical assistance request from a requester may include: identifying an incident type for the medical assistance request. Assigning an autonomous drone to the medical assistance request, thus defining an assigned autonomous drone may include: assigning an autonomous drone to the medical assistance request based, at least in part, upon the incident type. The assigned autonomous drone may be configured to transport a medical professional to the incident location. The assigned autonomous drone may be configured to search the incident location for a subject of the medical assistance request. The assigned autonomous drone may be configured to transport a subject of the medical assistance request to a medical facility. The assigned autonomous drone may be configured to communicate with a medical facility. The assigned autonomous drone may be configured to communicate with a subject of the medical assistance request.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes a drone navigation process according to an embodiment of the present disclosure;

FIGS. 2A-2E are diagrammatic views of an autonomous drone for use with the drone navigation process of FIG. 1 according to an embodiment of the present disclosure;

FIG. 3 is a flowchart of the drone navigation process of FIG. 1 according to an embodiment of the present disclosure; and

FIG. 4 is a flowchart of the drone navigation process of FIG. 1 according to an embodiment of the present disclosure.

Like reference symbols in the various drawings indicate like elements.

### DETAILED DESCRIPTION OF THE PREFERRED EMBODIMENTS

System Overview

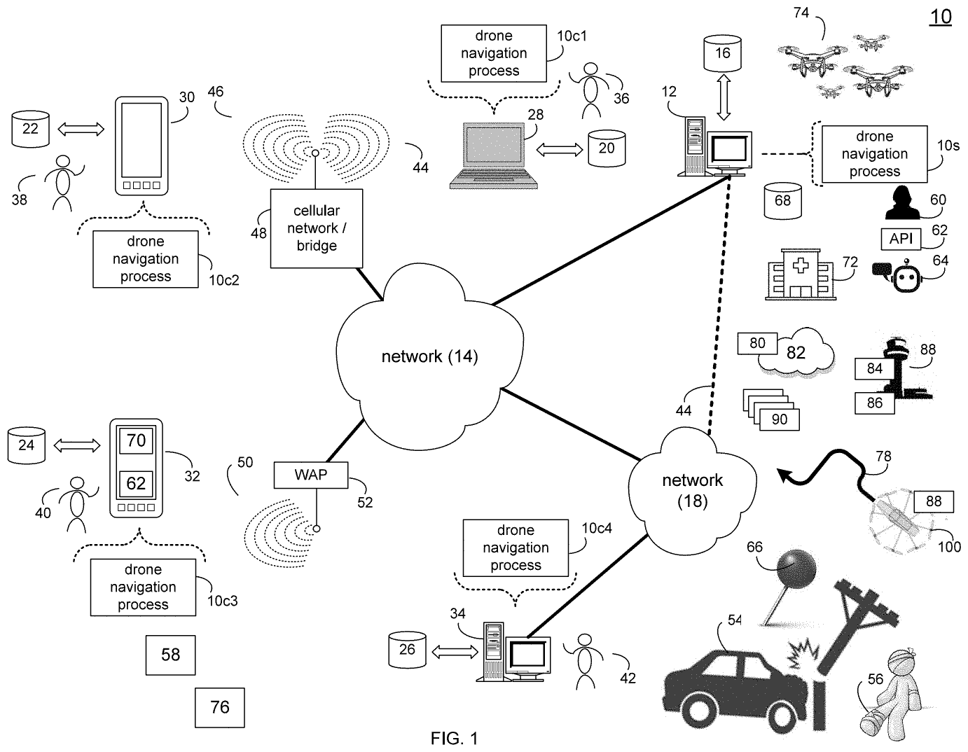

Referring to FIG. 1, there is shown drone navigation process 10. Drone navigation process 10 may be implemented as a server-side process, a client-side process, or a hybrid server-side/client-side process. For example, drone navigation process 10 may be implemented as a purely server-side process via drone navigation process 10s. Alternatively, drone navigation process 10 may be implemented as a purely client-side process via one or more of drone navigation process 10c1, drone navigation process 10c2, drone navigation process 10c3, and drone navigation process 10c4. Alternatively still, drone navigation process 10 may be implemented as a hybrid server-side/client-side process via drone navigation process 10s in combination with one or more of drone navigation process 10c1, drone navigation process 10c2, drone navigation process 10c3, and drone navigation process 10c4. Accordingly, drone navigation process 10 as used in this disclosure may include any combination of drone navigation process 10s, drone navigation process 10c1, drone navigation process 10c2, drone navigation process 10c3, and drone navigation process 10c4.

Drone navigation process 10s may be a server application and may reside on and may be executed by computing device 12, which may be connected to network 14 (e.g., the Internet or a local area network). Examples of computing device 12 may include, but are not limited to: a personal computer, a server computer, a series of server computers, a mini computer, a mainframe computer, a smartphone, or a cloud-based computing platform.

The instruction sets and subroutines of drone navigation process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of drone navigation processes 10c1, 10c2, 10c3, 10c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of drone navigation processes 10c1, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; RAID devices; random access memories (RAM); read-only memories (ROM), and all forms of flash memory storage devices.

Examples of client electronic devices 28, 30, 32, 34 may include, but are not limited to a personal digital assistant (not shown), a tablet computer (not shown), laptop computer 28, smart phone 30, smart phone 32, personal computer 34, a notebook computer (not shown), a server computer (not shown), a gaming console (not shown), and a dedicated network device (not shown). Client electronic devices 28, 30, 32, 34 may each execute an operating system, examples of which may include but are not limited to Microsoft Windows™, Android™, iOS™, Linux™, or a custom operating system.

Users 36, 38, 40, 42 may access drone navigation process 10 directly through network 14 or through secondary network 18. Further, drone navigation process 10 may be connected to network 14 through secondary network 18, as illustrated with link line 44.

The various client electronic devices (e.g., client electronic devices 28, 30, 32, 34) may be directly or indirectly coupled to network 14 (or network 18). For example, laptop computer 28 and smart phone 30 are shown wirelessly coupled to network 14 via wireless communication channels 44, 46 (respectively) established between laptop computer 28, smart phone 30 (respectively) and cellular network/bridge 48, which is shown directly coupled to network 14. Further, smart phone 32 is shown wirelessly coupled to network 14 via wireless communication channel 50 established between smart phone 32 and wireless access point (i.e., WAP) 52, which is shown directly coupled to network 14. Additionally, personal computer 34 is shown directly coupled to network 18 via a hardwired network connection.

WAP 52 may be, for example, an IEEE 802.11a, 802.11b, 802.11g, 802.11n, Wi-Fi, and/or Bluetooth device that is capable of establishing wireless communication channel 50 between smart phone 32 and WAP 52. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

Autonomous Drone

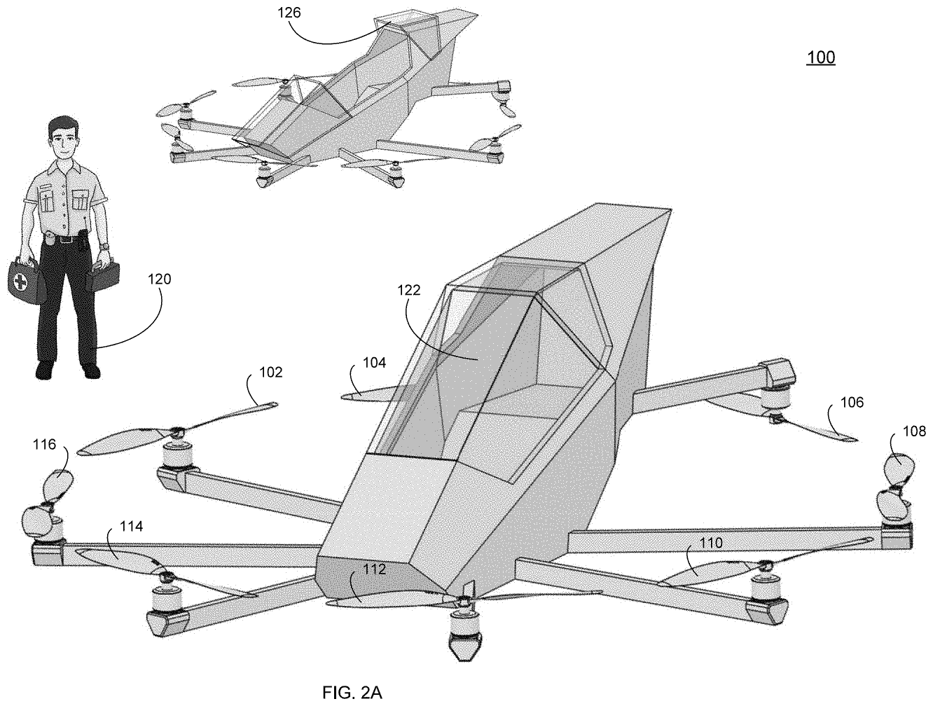

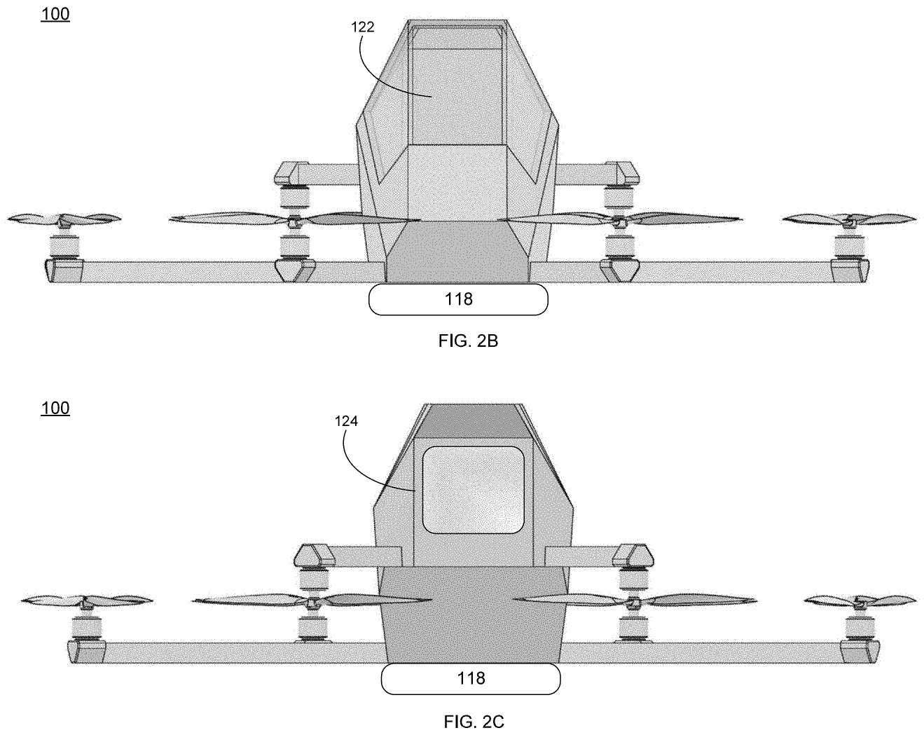

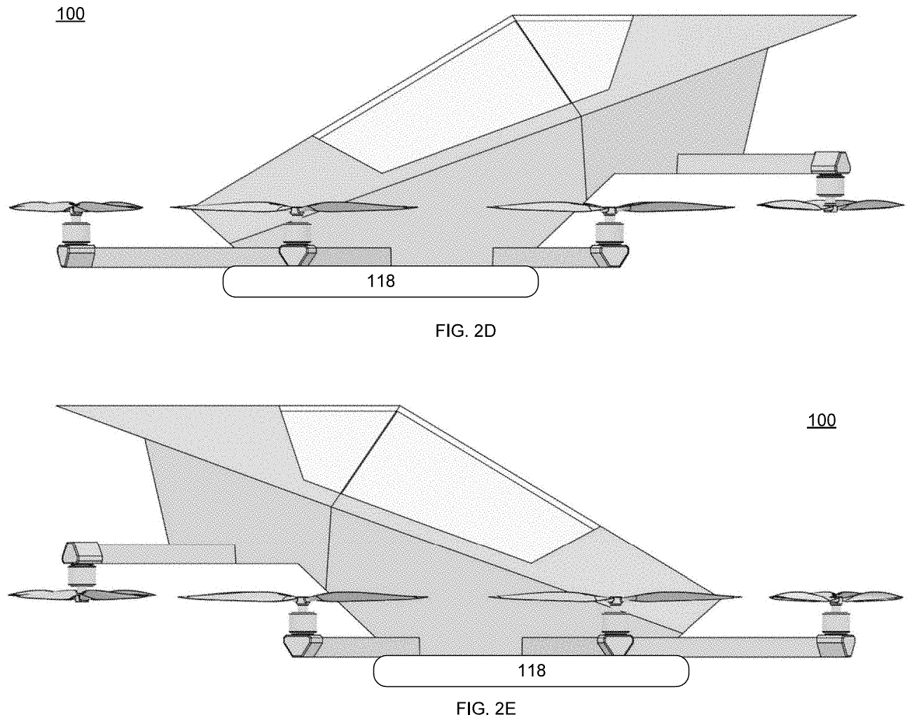

Referring to FIGS. 2A-2E, there is shown autonomous drone 100. As is known in the art, an autonomous drone is a type of unmanned aerial vehicle (UAV) that is capable of operating without the need for direct human input or control. These drones can be programmed with pre-set flight paths and instructions, allowing them to navigate through an environment and complete specific tasks autonomously. As used in this disclosure, autonomous drone (e.g., autonomous drone 100) is intended to mean any drone that is capable of self-navigating (regardless of whether or not it is carrying people or payloads). Accordingly, one example of autonomous drone 100 may be a self driving “air cab” that auto-navigates in an unoccupied state to a pickup location, picks up a passenger, auto-navigates in an occupied state to a destination location, drops off the passenger, and then auto-navigates in an unoccupied state to another pickup location. Autonomous drones typically use a combination of sensors, software, and onboard computing power to navigate and make decisions. They may use GPS and other location-based technologies to determine their position and avoid collisions with obstacles or other objects. Some autonomous drones may also use machine learning or artificial intelligence algorithms to analyze data and make decisions based on their environment. Autonomous drones have a wide range of potential applications, including aerial photography and video, surveillance and security, scientific research, agriculture, and package delivery. They offer several advantages over traditional manned aircraft, including increased safety, reduced costs, and improved efficiency.

Autonomous drone 100 may include a plurality of rotors (e.g., rotors 102, 104, 106, 108, 110, 112, 114, 116). While in this particular example, autonomous drone 100 is shown to include eight rotors (e.g., rotors 102, 104, 106, 108, 110, 112, 114, 116), this is for illustrative purposes only and is not intended to be a limitation of this disclosure, as other configurations are possible and are considered to be within the scope of this disclosure. For example, the number of rotors may be increased or decreased depending upon the specific needs of the drone. Through the use of the plurality of rotors (e.g., rotors 102, 104, 106, 108, 110, 112, 114, 116), the roll axis, pitch axis and yaw axis of autonomous drone 100 may be controlled.

If autonomous drone 100 is to be used for search and rescue operations, autonomous drone 100 may be configured to search a location for people in need of assistance. For example, autonomous drone 100 may include thermal imagining camera 118 to effectuate such searching operations.

As is known in the art, thermal imagining camera 118 (also known as a thermographic camera) is a type of camera that captures images of the heat emitted by objects in the environment. These cameras are capable of detecting the infrared radiation emitted by objects and converting it into a visible image that shows the variations in temperature across the scene. Thermal imaging cameras are widely used in a variety of applications, including industrial and commercial inspections, firefighting, medical imaging, and military surveillance. They are particularly useful in applications where traditional cameras cannot provide useful information, such as in complete darkness, in fog or smoke, or in areas with poor visibility.

Thermal imaging cameras work by detecting the heat signatures of objects and converting them into a visual image. The images produced by these cameras are typically displayed in a range of colors, with hotter areas appearing as red, orange, or yellow, and cooler areas appearing as blue, purple, or black. This allows operators to quickly identify areas of interest and potential problems. Thermal imaging cameras are available in a range of sizes and configurations, from handheld devices to larger systems that are mounted on drones, vehicles, or buildings.

Automated Search/Rescue/Ambulatory

Assume for the following example that autonomous drone 100 is configured to perform search/rescue/ambulatory services. Further, assume that user 40 witnesses the occurrence of car accident 54, wherein driver 56 was injured. Accordingly, user 40 may request an autonomous drone (e.g., autonomous drone 100) to assist injured driver 56, resulting in the generation of medical assistance request 58.

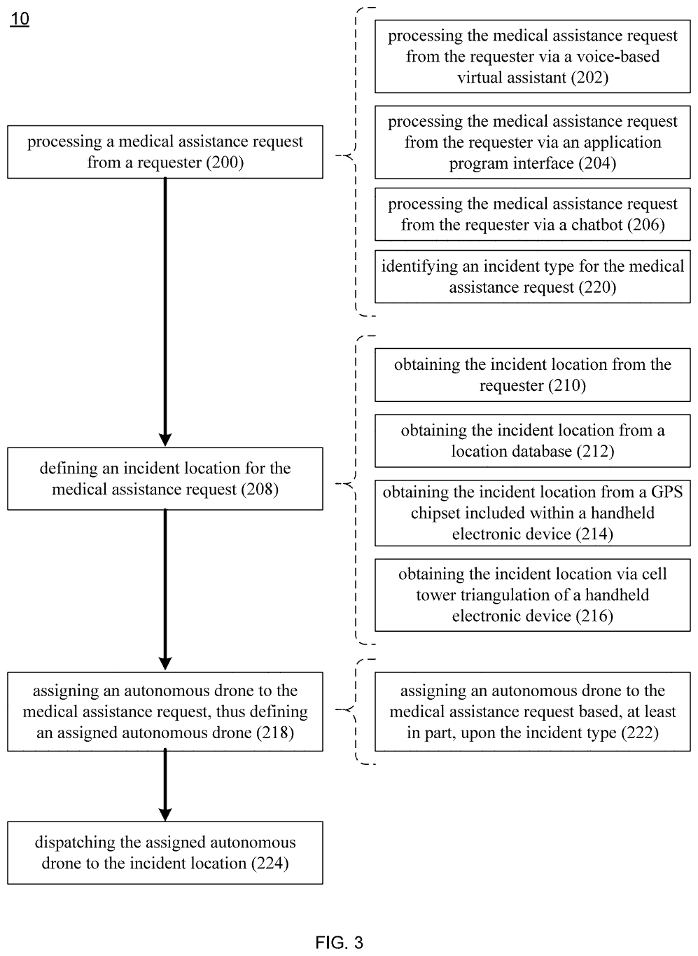

Referring also to FIG. 3, upon receipt of medical assistance request 58, drone navigation process 10 may process 200 the medical assistance request (e.g., medical assistance request 58) from the requester (e.g., user 40).

When processing 200 the medical assistance request (e.g., medical assistance request 58) from the requester (e.g., user 40), drone navigation process 10 may process 202 the medical assistance request (e.g., medical assistance request 58) from the requester (e.g., user 40) via voice-based virtual assistant 60 (e.g., if medical assistance request 58 is a voice-based request) or via a human operator (not shown).

As is known in the art, a virtual assistant is an AI-powered software application that can perform various tasks and services for users. Virtual assistants are designed to mimic human interactions and provide personalized assistance to users through natural language processing and machine learning algorithms. Virtual assistants can perform a wide range of tasks, including scheduling appointments, setting reminders, sending messages, making phone calls, ordering food, providing weather updates, answering questions, and even playing music or videos. Virtual assistants are commonly integrated into popular mobile devices, smart speakers, and other internet-connected devices, and can be accessed through voice commands or through text-based chat interfaces. Some examples of popular virtual assistants include Apple's Siri, Amazon's Alexa, Google Assistant, and Microsoft's Cortana. Virtual assistants have become increasingly popular in recent years as more people rely on technology to help them manage their daily tasks and activities.

When processing 200 the medical assistance request (e.g., medical assistance request 58) from the requester (e.g., user 40), drone navigation process 10 may process 204 the medical assistance request (e.g., medical assistance request 58) from the requester (e.g., user 40) via application program interface 62 (e.g., if medical assistance request 58 is initiated via application 62 executed on smart phone 32).

As is known in the art, an Application Programming Interface (API) is a set of protocols, routines, and tools that enable software developers to build software applications that can interact with other software components or services. APIs provide a standardized way for developers to access and use the functionality of another system without needing to know the underlying details of how it works. This enables different software systems to communicate with each other, exchange data, and perform various operations. APIs can take various forms, including web APIs that enable communication over the internet and operating system APIs that provide access to system-level functionality. APIs can also be classified into public or private, depending on whether they are intended for general use or restricted to specific users or organizations. APIs play a critical role in modern software development, and they are used in a wide range of applications, from mobile apps to web applications to enterprise systems. They enable developers to build more robust, scalable, and interoperable applications that can communicate and exchange data with other systems seamlessly.

When processing 200 the medical assistance request (e.g., medical assistance request 58) from the requester (e.g., user 40), drone navigation process 10 may process 206 the medical assistance request (e.g., medical assistance request 58) from the requester (e.g., user 40) via chatbot 64.

As is known in the art, a chatbot is a software program that uses artificial intelligence (AI) and natural language processing (NLP) to simulate human conversation through text interactions. Chatbots are designed to mimic human communication and provide personalized assistance to users, often in the form of automated customer service. Chatbots can be integrated into websites, messaging apps, or social media platforms, allowing users to interact with them through chat interfaces. Chatbots can perform a wide range of tasks, such as answering frequently asked questions, providing customer support, booking appointments, making reservations, and even providing recommendations. Chatbots use machine learning algorithms to understand and interpret user inputs, allowing them to respond appropriately and provide relevant information. They can also learn from user interactions over time, becoming more accurate and effective in their responses. Chatbots have become increasingly popular in recent years as more businesses adopt them to improve their customer service and streamline their operations.

Upon processing 200 medical assistance request 58 from user 40, drone navigation process 10 may define 208 an incident location (e.g., incident location 66) for the medical assistance request (e.g., medical assistance request 58).

When defining 208 an incident location (e.g., incident location 66) for the medical assistance request (e.g., medical assistance request 58), drone navigation process 10 may obtain 210 the incident location (e.g., incident location 66) from the requester (e.g., user 40). For example and if medical assistance request 58 is a voice-based request, user 40 may provide voice-based virtual assistant 60 with incident location 66. Alternatively, user 40 may provide incident location 66 to a human operator (not shown).

When defining 208 an incident location (e.g., incident location 66) for the medical assistance request (e.g., medical assistance request 58), drone navigation process 10 may obtain 212 the incident location (e.g., incident location 66) from a location database (e.g., 911 database 68).

As is known in the art, a 911 database 68 is a database system used by emergency response services that associates phone numbers with physical locations. Accordingly, when a call comes in from a specific phone number, the location of that phone number may be obtained from such a database.

When defining 208 an incident location (e.g., incident location 66) for the medical assistance request (e.g., medical assistance request 58), drone navigation process 10 may obtain 214 the incident location (e.g., incident location 66) from a GPS chipset (e.g., GPS chipset 70) included within a handheld electronic device (e.g., smartphone 32).

As is known in the art, a GPS chipset (e.g., GPS chipset 70) is a specialized integrated circuit that is used to receive, process, and decode signals from GPS (Global Positioning System) satellites. The GPS chipset (e.g., GPS chipset 70) is an essential component of GPS-enabled devices such as smartphones, smartwatches, and navigation systems. The GPS chipset (e.g., GPS chipset 70) includes multiple components, such as a receiver, an antenna, and a processor. The receiver captures the GPS signals transmitted by satellites, while the antenna helps to amplify and filter the signals. The processor then decodes the GPS signals and uses them to determine the device's location.

When defining 208 an incident location (e.g., incident location 66) for the medical assistance request (e.g., medical assistance request 58), drone navigation process 10 may obtain 216 the incident location (e.g., incident location 66) via cell tower triangulation of a handheld electronic device (e.g., smartphone 32).

As is known in the art, cell tower triangulation is a technique used to determine the approximate location of a mobile device (e.g., smartphone 32) by using the signal strength of nearby cell towers (not shown). This technique is often used when GPS or other location-based services are unavailable or inaccurate. When a mobile device (e.g., smartphone 32) is in range of one or more cell towers, it sends and receives signals to establish a connection to the cellular network. Each cell towers (not shown) has a unique identification number and a known geographic location. By measuring the signal strength and timing of the signals received from different cell towers (not shown), the location of the mobile device (e.g., smartphone 32) can be estimated using triangulation.

Drone navigation process 10 may assign 218 an autonomous drone (e.g., autonomous drone 100) to the medical assistance request (e.g., medical assistance request 58), thus defining an assigned autonomous drone (e.g., autonomous drone 100).

For example and when processing 200 a medical assistance request (e.g., medical assistance request 58) from a requester (e.g., user 40), drone navigation process 10 may identify 220 an incident type for the medical assistance request (e.g., medical assistance request 58). Examples of such an incident type may include but are not limited to: a car accident event; a cardiac event, a burn event, etc. Accordingly, medical assistance request 58 may define such an incident type.

When assigning 218 an autonomous drone (e.g., autonomous drone 100) to the medical assistance request (e.g., medical assistance request 58), thus defining an assigned autonomous drone (e.g., autonomous drone 100), drone navigation process 10 may assign 222 an autonomous drone (e.g., autonomous drone 100) to the medical assistance request (e.g., medical assistance request 58) based, at least in part, upon the incident type.

For example:
if the incident type is a car accident event, drone navigation process 10 may assign 222 an autonomous drone (e.g., autonomous drone 100) to the medical assistance request (e.g., medical assistance request 58) that is configured to stabilize/triage accident victims (e.g., via splints, neck collars, etc.);
if the incident type is a cardiac event, drone navigation process 10 may assign 222 an autonomous drone (e.g., autonomous drone 100) to the medical assistance request (e.g., medical assistance request 58) that is configured to stabilize a heart attack victim (e.g., via defibrillation equipment, EKG equipment, etc.); and
if the incident type is a burn event, drone navigation process 10 may assign 222 an autonomous drone (e.g., autonomous drone 100) to the medical assistance request (e.g., medical assistance request 58) that is configured to stabilize burn victims (e.g., via creams, gauze, etc.).

Once assigned 218, drone navigation process 10 may dispatch 224 the assigned autonomous drone (e.g., autonomous drone 100) to the incident location (e.g., incident location 66).

The assigned autonomous drone (e.g., autonomous drone 100) may be configured to:
transport a medical professional (e.g., medical professional 120) to the incident location (e.g., incident location 66) via cabin 122;
search the incident location (e.g., incident location 66) for a subject (e.g., injured driver 56) of the medical assistance request (e.g., medical assistance request 58) via thermal imagining camera 118;
transport a subject (e.g., injured driver 56) of the medical assistance request (e.g., medical assistance request 58) to a medical facility (e.g., hospital 72) via transport bay 124 or opening canopy 126;
communicate with a medical facility (e.g., hospital 72) to provide status information (e.g., vital signs) of injured driver 56; and
communicate with a subject (e.g., injured driver 56) of the medical assistance request (e.g., medical assistance request 58).

Automated Navigation

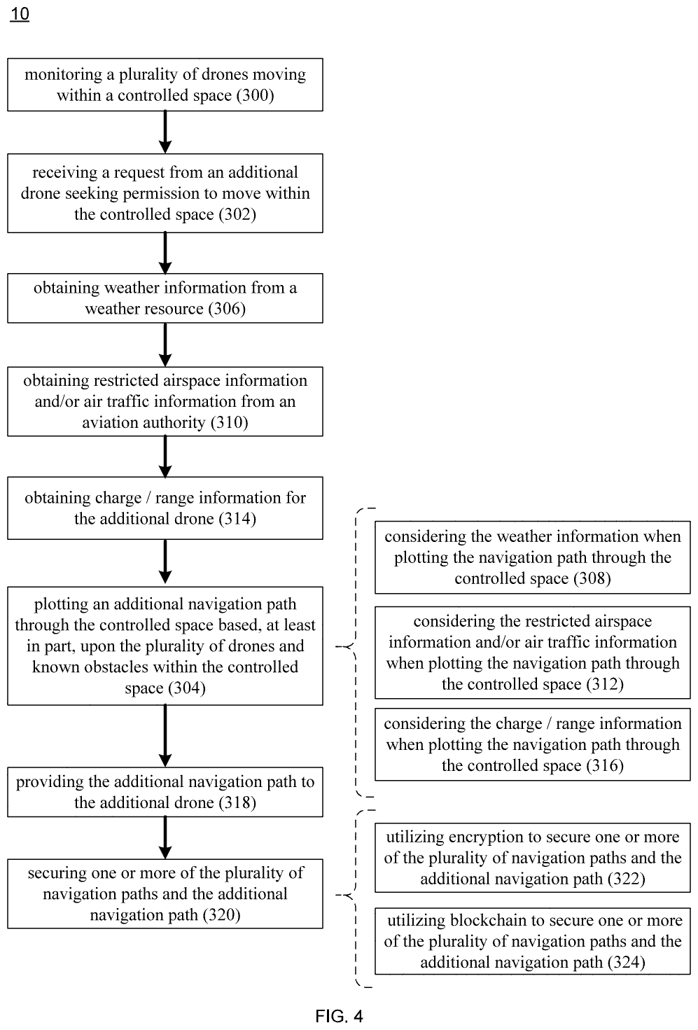

Referring also to FIG. 4, assume for the following example that autonomous drone 100 is configured to transport people between locations. Further, assume that user 40 wishes to travel from a first location to a second location across town. Accordingly, user 40 may request that an autonomous drone (e.g., autonomous drone 100) transport user 40 from the first location to the second location. According and in order to effectuate the safe use/travel of such autonomous drones, drone navigation process 10 may monitor 300 a plurality of drones (e.g., plurality of drones 74) moving within a controlled space. Examples of such a controlled space may include but are not limited to: the air space of a town, a city, a state or a country.

Accordingly and in the situation in which user 40 requests that autonomous drone 100 transport them from the first location to the second location, drone navigation process 10 may receive 302 a request (e.g., transportation request 76) from an additional drone (e.g., autonomous drone 100) seeking permission to move within the controlled space (e.g., the air space between the first location and the second location).

Accordingly, drone navigation process 10 may plot 304 an additional navigation path (e.g., navigation path 78) through the controlled space based, at least in part, upon the plurality of drones (e.g., plurality of drones 74) and known obstacles (e.g., buildings, bridges, mountains, monuments, etc.) within the controlled space. As would be expected, navigation path 78 may define various directions, altitudes, velocities, etc. Specifically, navigation path 78 may be plotted 304 by drone navigation process 10 to navigate autonomous drone 100 from the first location and the second location while avoiding each of plurality of drones 74 and any obstacles (e.g., buildings, bridges, mountains, monuments, etc.) within the controlled space.

Additionally, drone navigation process 10 may obtain 306 weather information 80 from weather resource 82 (e.g., the National Weather Service). When plotting 304 an additional navigation path (e.g., navigation path 78) through the controlled space based, at least in part, upon the plurality of drones (e.g., plurality of drones 74) and known obstacles within the controlled space, drone navigation process 10 may consider 308 weather information 80 when plotting the additional navigation path (e.g., navigation path 78) through the controlled space. Accordingly, navigation path 78 may plot around bad/undesirable weather.

Further, drone navigation process 10 may obtain 310 restricted airspace information 84 and/or air traffic information 86 from aviation authority 88 (e.g., the Federal Aviation Authority). When plotting 304 an additional navigation path (e.g., navigation path 78) through the controlled space based, at least in part, upon the plurality of drones (e.g., plurality of drones 74) and known obstacles within the controlled space, drone navigation process 10 may consider 312 restricted airspace information 84 and/or air traffic information 86 when plotting the additional navigation path (e.g., navigation path 78) through the controlled space. Accordingly, navigation path 78 may plot around restricted airspace (e.g., airports, military bases, etc.) and commercial/civilian/military aircraft.

Also, drone navigation process 10 may obtain 314 charge/range information 88 for the additional drone (e.g., autonomous drone 100). When plotting 304 an additional navigation path (e.g., navigation path 78) through the controlled space based, at least in part, upon the plurality of drones (e.g., plurality of drones 74) and known obstacles within the controlled space, drone navigation process 10 may consider 316 the charge/range information 88 when plotting the additional navigation path (e.g., navigation path 78) through the controlled space. Accordingly, navigation path 78 only define a path that autonomous drone 100 has sufficient charge/range to complete.

Drone navigation process 10 may provide 318 the additional navigation path (e.g., navigation path 78) to the additional drone (e.g., autonomous drone 100), which may be utilized to navigate autonomous drone 100 from the first location to the second location.

As could be imagined, each of the plurality of drones (e.g., plurality of drones 74) moving within a controlled space has a defined navigation path that enables each of the drones to reach their destination, thus defining a plurality of navigation paths (e.g., plurality of navigation paths 90).

Accordingly and in order to protect autonomous drones from being hacked/taken over/reprogrammed, drone navigation process 10 may secure 320 one or more of the plurality of navigation paths (e.g., plurality of navigation paths 90) and the additional navigation path (e.g., navigation path 78).

When securing 320 one or more of the plurality of navigation paths (e.g., plurality of navigation paths 90) and the additional navigation path (e.g., navigation path 78), drone navigation process 10 may utilize 322 data encryption to secure one or more of the plurality of navigation paths (e.g., plurality of navigation paths 90) and the additional navigation path (e.g., navigation path 78).

As is known in the art, data encryption is the process of converting plain, readable data into a coded or encrypted form to secure it from unauthorized access or interception. Encryption involves using an algorithm or cipher to transform the original data (also known as plaintext) into a form that is not easily readable without a decryption key or password. The encrypted data, also known as ciphertext, appears as a jumbled sequence of letters, numbers, and symbols, making it difficult to decipher and read. Encryption is used to protect sensitive information such as passwords, financial data, and personal information, especially when it is being transmitted over insecure networks such as the internet. There are several types of encryption algorithms used to secure data, including symmetric key encryption, asymmetric key encryption, and hashing. Symmetric key encryption uses the same key to encrypt and decrypt data, while asymmetric key encryption uses a pair of public and private keys. Hashing involves generating a unique fixed-length code that represents the original data and cannot be reversed to reveal the original data. Accordingly and through the use of such data encryption, the navigation paths being travelled by these autonomous drones may be protected from attack/hacking, thus minimizing the likelihood of the autonomous drones from being taken over/reprogrammed.

When securing 320 one or more of the plurality of navigation paths (e.g., plurality of navigation paths 90) and the additional navigation path (e.g., navigation path 78), drone navigation process 10 may utilize 324 blockchain technology to secure one or more of the plurality of navigation paths (e.g., plurality of navigation paths 90) and the additional navigation path (e.g., navigation path 78).

As is known in the art, blockchain technology is a decentralized digital ledger technology that allows for secure, transparent, and tamper-proof transactions and record-keeping. In a blockchain network, transactions are recorded in a block, which is then added to a chain of previously recorded blocks, forming a permanent and unalterable record. The most well-known use case of blockchain technology is in cryptocurrencies like Bitcoin, where the blockchain is used to keep track of all transactions on the network. However, blockchain has many other potential applications beyond cryptocurrencies, including supply chain management, voting systems, and digital identity verification. One of the key features of blockchain technology is that it is decentralized, meaning there is no central authority controlling the network. Instead, all participants in the network have a copy of the ledger, and transactions are validated and recorded through a consensus mechanism. Accordingly and through the use of such blockchain technology, the navigation paths being travelled by these autonomous drones may be protected from attack/hacking, thus minimizing the likelihood of the autonomous drones from being taken over/reprogrammed.

General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method executed on a computing device comprising:
processing a medical assistance request from a requester;
defining an incident location for the medical assistance request;
assigning an autonomous drone to the medical assistance request, thus defining an assigned autonomous drone; and
dispatching the assigned autonomous drone to the incident location.

2. The computer-implemented method of claim 1 wherein processing a medical assistance request from a requester includes one or more of:
processing the medical assistance request from the requester via a voice-based virtual assistant;
processing the medical assistance request from the requester via an application program interface; and
processing the medical assistance request from the requester via a chatbot.

3. The computer-implemented method of claim 1 wherein defining an incident location for the medical assistance request includes one or more of:
obtaining the incident location from the requester;
obtaining the incident location from a location database;
obtaining the incident location from a GPS chipset included within a handheld electronic device; and
obtaining the incident location via cell tower triangulation of a handheld electronic device.

4. The computer-implemented method of claim 1 wherein processing a medical assistance request from a requester includes:
identifying an incident type for the medical assistance request.

5. The computer-implemented method of claim 1 wherein assigning an autonomous drone to the medical assistance request, thus defining an assigned autonomous drone includes:
assigning an autonomous drone to the medical assistance request based, at least in part, upon the incident type.

6. The computer-implemented method of claim 1 wherein the assigned autonomous drone is configured to transport a medical professional to the incident location.

7. The computer-implemented method of claim 1 wherein the assigned autonomous drone is configured to search the incident location for a subject of the medical assistance request.

8. The computer-implemented method of claim 1 wherein the assigned autonomous drone is configured to transport a subject of the medical assistance request to a medical facility.

9. The computer-implemented method of claim 1 wherein the assigned autonomous drone is configured to communicate with a medical facility.

10. The computer-implemented method of claim 1 wherein the assigned autonomous drone is configured to communicate with a subject of the medical assistance request.

11. A computer program product residing on a computer readable medium having a plurality of instructions stored thereon which, when executed by a processor, cause the processor to perform operations comprising:
processing a medical assistance request from a requester;
defining an incident location for the medical assistance request;
assigning an autonomous drone to the medical assistance request, thus defining an assigned autonomous drone; and
dispatching the assigned autonomous drone to the incident location.

12. The computer program product of claim 11 wherein processing a medical assistance request from a requester includes one or more of:
processing the medical assistance request from the requester via a voice-based virtual assistant;
processing the medical assistance request from the requester via an application program interface; and
processing the medical assistance request from the requester via a chatbot.

13. The computer program product of claim 11 wherein defining an incident location for the medical assistance request includes one or more of:
obtaining the incident location from the requester;
obtaining the incident location from a location database;
obtaining the incident location from a GPS chipset included within a handheld electronic device; and
obtaining the incident location via cell tower triangulation of a handheld electronic device.

14. The computer program product of claim 11 wherein processing a medical assistance request from a requester includes:
identifying an incident type for the medical assistance request.

15. The computer program product of claim 11 wherein assigning an autonomous drone to the medical assistance request, thus defining an assigned autonomous drone includes:
assigning an autonomous drone to the medical assistance request based, at least in part, upon the incident type.

16. The computer program product of claim 11 wherein the assigned autonomous drone is configured to transport a medical professional to the incident location.

17. The computer program product of claim 11 wherein the assigned autonomous drone is configured to search the incident location for a subject of the medical assistance request.

18. The computer program product of claim 11 wherein the assigned autonomous drone is configured to transport a subject of the medical assistance request to a medical facility.

19. The computer program product of claim 11 wherein the assigned autonomous drone is configured to communicate with a medical facility.

20. The computer program product of claim 11 wherein the assigned autonomous drone is configured to communicate with a subject of the medical assistance request.

21. A computing system including a processor and memory configured to perform operations comprising:
processing a medical assistance request from a requester;
defining an incident location for the medical assistance request;
assigning an autonomous drone to the medical assistance request, thus defining an assigned autonomous drone; and
dispatching the assigned autonomous drone to the incident location.

22. The computing system of claim 21 wherein processing a medical assistance request from a requester includes one or more of:
processing the medical assistance request from the requester via a voice-based virtual assistant;
processing the medical assistance request from the requester via an application program interface; and
processing the medical assistance request from the requester via a chatbot.

23. The computing system of claim 21 wherein defining an incident location for the medical assistance request includes one or more of:
obtaining the incident location from the requester;
obtaining the incident location from a location database;
obtaining the incident location from a GPS chipset included within a handheld electronic device; and
obtaining the incident location via cell tower triangulation of a handheld electronic device.

24. The computing system of claim 21 wherein processing a medical assistance request from a requester includes:
identifying an incident type for the medical assistance request.

25. The computing system of claim 21 wherein assigning an autonomous drone to the medical assistance request, thus defining an assigned autonomous drone includes:
assigning an autonomous drone to the medical assistance request based, at least in part, upon the incident type.

26. The computing system of claim 21 wherein the assigned autonomous drone is configured to transport a medical professional to the incident location.

27. The computing system of claim 21 wherein the assigned autonomous drone is configured to search the incident location for a subject of the medical assistance request.

28. The computing system of claim 21 wherein the assigned autonomous drone is configured to transport a subject of the medical assistance request to a medical facility.

29. The computing system of claim 21 wherein the assigned autonomous drone is configured to communicate with a medical facility.

30. The computing system of claim 21 wherein the assigned autonomous drone is configured to communicate with a subject of the medical assistance request.
